(19) United States
(12) Patent Application Publication
(10) Pub. No.: US 2020/0330433 A1
(43) Pub. Date: Oct. 22, 2020

(54) EXTENDED RELEASE PHARMACEUTICAL COMPOSITION OF APREMILAST

(71) Applicant: MANKIND PHARMA LTD., New Delhi (IN)

(72) Inventors: Vivek JAIN, Manesar, Gurugram (IN); Rajeshwar SANGWAI, Manesar, Gurugram (IN); P.V.S NARASIMHAM, Manesar, Gurugram (IN); Anil KUMAR, Manesar, Gurugram (IN)

(21) Appl. No.: 16/754,307

(22) PCT Filed: Oct. 1, 2018
(86) PCT No.: PCT/IN2018/050627

§ 371 Date: Apr. 7, 2020

(30) Foreign Application Priority Data
Oct. 10, 2017 (IN) 201711035901

(51) Int. Cl. A61K 31/4035; A61K 9/28

(52) U.S. Cl. CPC A61K 31/4035; A61K 9/2833

(57) ABSTRACT

The invention relates to an extended release oral pharmaceutical composition comprising a core, wherein the core comprises apremilast or pharmaceutically acceptable salts, ester, solvates, polymorphs thereof, at least one extended release polymer and one or more pharmaceutically acceptable excipients, wherein the core is optionally coated with a film coating composition. The composition of the invention can be used for the treatment of psoriasis, psoriatic arthritis.

### FIELD OF THE INVENTION

The present invention relates to an extended release oral pharmaceutical composition comprising a core, wherein the core comprises apremilast, at least one extended release polymer and one or more pharmaceutically acceptable excipients, wherein the core is optionally coated with a film coating composition.

### BACKGROUND OF THE INVENTION

The convenience of administering a single dose of medication which releases active ingredient over an extended period of time as opposed to the administration of a number of single doses at regular intervals has long been recognized in the pharmaceutical art. The advantage to the patient and clinician in having consistent and uniform blood levels of medication over an extended period of time are likewise recognized. In sustained release preparations, the dosage form continuously provide drug for absorption into the blood stream to replace the amount eliminated while the dosage form is passing through the gastrointestinal tract of the patient.

The conventional approaches to sustained release formulation can be disadvantageous in that certain classes of active ingredients are not suited to absorption during passage through the gastrointestinal tract due to their physiochemical properties and/or favourable sites of absorption. For example, most acidic medicaments are principally absorbed from the stomach, whereas most basic medicaments are absorbed primarily from the intestine. Most medicaments will undergo varying degrees of change in solubility by passage from the acutely acidic conditions of the stomach to the neutral to alkaline conditions of the intestine.

It is readily apparent in view of the above considerations that a large number of medicaments are not amenable to conventional sustained release formulations which are not retained in the stomach and which release medicament in the intestine. It is equally apparent that a sustained release formulation which is retained in the stomach and which slowly releases medicament in the stomach over an extended period of time would be eminently suited to such medicaments. Such a sustained release formulation is provided by the present invention.

Apremilast is a phosphodiesterase 4 (PDE4) inhibitor. Apremilast is chemically known as N-[2-[(1S)-1-(3-ethoxy-4-methoxyphenyl)-2-(methyl sulfonyl) ethyl]-2,3-dihydro-1,3-dioxo-1H-isoindol-4-yl] acetamide having Formula (I).

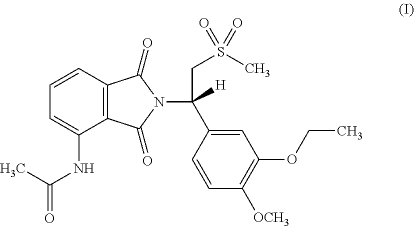 [SMILES: CCOc1cc([C@@H](CS(C)(=O)=O)N2C(=O)c3cccc(NC(C)=O)c3C2=O)ccc1OC]

Apremilast is indicated for the treatment of adult patients with active psoriatic arthritis. It is available under the trade name of Otezla® tablets supplied in 10, 20, and 30 mg strengths for oral administration. There are several references known in the literature, which describe the different polymorphic forms of apremilast and pharmaceutical composition of the same, which are used in the treatment of various diseases.

U.S. Pat. No. 6,020,358 discloses racemic apremilast and process for its preparation.

U.S. Pat. No. 6,962,940 discloses method of using stereomerically pure apremilast for the treatment of certain diseases or disorders including, for example, inflammation.

U.S. Pat. No. 7,208,516 discloses method of using apremilast for the treatment of psoriatic arthritis.

U.S. Pat. Nos. 7,659,302 & 8,455,536 discloses method of using “stereomerically pure” apremilast for the treatment of psoriasis.

U.S. Pat. No. 7,427,638 discloses stereomerically pure (+)-2-[1-(3-ethoxy-4-methoxyphenyl)-2-methylsulfonylethyl]-4-acetylaminoisoindoline-1,3-dione, substantially free of its (−)isomer, or a pharmaceutically acceptable metabolite, salt, solvate or hydrate, thereof and its pharmaceutical composition.

U.S. Pat. No. 8,802,717 discloses methods of using “stereomerically pure” apremilast for the treatment of arthritic conditions.

U.S. Pat. No. 9,018,243 discloses methods of using apremilast Form B, including, for example, the treatment of psoriatic arthritis and/or psoriasis.

U.S. Pat. No. 8,629,173 discloses methods of using apremilast Form A & F, including, for example, the treatment of psoriasis.

U.S. Pat. No. 9,351,957 discloses an amorphous form of apremilast and pharmaceutical composition comprising amorphous apremilast.

U.S. Pat. No. 9,532,977 discloses a controlled release oral dosage form comprising apremilast, a swelling excipient; poly (butyl methacylate-co-2-dimethylaminoethyl methacrylate-co-methyl methacrylate)(1:2:1); an anionic polymer in acidic pH, and sodium bicarbonate.

US Pub. No. 2016/0045475 discloses a method of treating psoriasis comprising escalating doses of stereomerically pure apremilast, wherein a starting dose is between about 10 mg per day and about 20 mg per day, and a maximum dose is between about 40 mg per day and about 100 mg per day. This seems to cover approved dosage regimen of Otezla® for psoriasis.

US Pub. No. 2014/0301980 discloses a method of treating psoriatic arthritis comprising escalating doses of stereomerically pure apremilast, wherein a starting dose is between about 10 mg per day and about 20 mg per day, and a maximum dose is between about 40 mg per day and about 100 mg per day. This seems to covers approved dosage regimen of Otezla® for psoriatic arthritis.

As pharmaceutical excipients have various functions and contribute to the pharmaceutical formulation in many different ways, e.g., solubilization, dilution, thickening, stabilization, preservation, coloring and flavoring. The properties that are commonly considered when formulating an active drug substance include bioavailability, ease of manufacture, ease of administration, and stability of the dosage form. Due to the varying properties of the active drug substance to be formulated, dosage forms typically require pharmaceutical excipients that are uniquely tailored for the active drug substance in order to achieve advantageous physical and pharmaceutical properties.

In view of the above, it is therefore, desirable to provide an extended release pharmaceutical composition of apremilast which are stable, bioavailable, has good release profile and as well as has improved patient compliance. Further, there exists a need to develop a process for the development of pharmaceutical composition which provides an efficient and more economical process.

### SUMMARY OF THE INVENTION

The present invention provides an extended release dosage form of apremilast comprising one or more extended release polymer and one or more pharmaceutically acceptable excipients and process of preparation thereof.

One embodiment of the present invention provides an extended release oral pharmaceutical composition comprising: (i) a core comprising apremilast or its pharmaceutically acceptable salts, ester, solvates, polymorphs thereof, one or more extended release polymer and one or more pharmaceutically acceptable excipients; and (ii) optionally a film coating.

Another embodiment of the present invention provides an extended release oral pharmaceutical composition comprising (i) a core comprising apremilast or pharmaceutically acceptable salts, ester, solvates, polymorphs thereof, one or more extended release polymer and one or more pharmaceutically acceptable excipients; and (ii) optionally a film coating comprising film forming polymer and one or more pharmaceutically acceptable excipients, wherein said pharmaceutically acceptable excipients are selected from the group comprising of diluent/filler, binder, disintegrant, surfactant, glidant, lubricant, film forming polymer, coloring agent/colorant, opacifier, plasticizer and/or combinations thereof.

Another embodiment of the present invention provides an extended release oral pharmaceutical composition comprising (i) a core comprising apremilast embedded in a matrix with one or more extended release polymer and one or more pharmaceutically acceptable excipients; and (ii) optionally a film coating.

Another embodiment of the present invention provides an extended release oral pharmaceutical composition comprising (i) a core comprising apremilast coated with one or more extended release polymer and one or more pharmaceutically acceptable excipients; and (ii) optionally a film coating over the core.

Another embodiment of the present invention provides an extended release oral pharmaceutical composition comprising (i) a core comprising apremilast and one or more pharmaceutically acceptable excipients, wherein said core is coated with an extended release coating comprising one or more extended release polymer and one or more pharmaceutically acceptable excipients; and (ii) optionally a film coating.

Another embodiment of the present invention provides an extended release oral pharmaceutical composition characterised by about 10-30% of apremilast is released within 2 hours; about 40-80% of apremilast is released within 8 hours and up to 95% of apremilast is released within 24 hours.

Another embodiment of the present invention provides an extended release oral pharmaceutical composition characterised by about 10-30% of apremilast is released within 2 hours; about 50-75% of apremilast is released within 8 hours and up to 95% of apremilast is released within 24 hours.

Another embodiment of the present invention provides a method of treating or preventing a disease or disorder ameliorated by the inhibition of TNF-[α] production, wherein the method comprises administering a therapeutically or prophylactically effective amount of an extended release oral pharmaceutical composition comprising a core comprising apremilast or pharmaceutically acceptable salts, ester, solvates, polymorphs thereof, at least one extended release polymer and one or more pharmaceutically acceptable excipients, wherein the core is optionally coated with a film coating composition.

Another embodiment of the present invention provides a process for preparation of extended release oral pharmaceutical composition of apremilast, wherein the process comprising the steps of (a) blending apremilast, one or more extended release polymer and one or more pharmaceutically acceptable excipients and (b) optionally film coating with coating material.

The details of one or more embodiments of the inventions are set forth in the description below. Other features, objects and advantages of the inventions will be apparent from the description.

### DESCRIPTION OF THE INVENTION

The present invention relates to an extended release oral pharmaceutical composition comprising a core comprising apremilast or pharmaceutically acceptable salts, ester, solvates, polymorphs thereof, at least one extended release polymer and one or more pharmaceutically acceptable excipients, optionally coated with film coating.

The term “active ingredient” herein refers to a pharmaceutically active molecule as well as its pharmaceutically acceptable and therapeutically active salts, esters, amides, prodrugs, metabolites, enantiomers, polymorphs, analogues, etc. that induce a desired pharmacological or physiological effect. Terms like “active”, “active agent”, “active substance” may be used synonymously for “active ingredient”.

The term “therapeutically effective amount” or “effective amount” used interchangeably, is defined to mean the amount or quantity of the active drug, which is sufficient to elicit an appreciable biological response when administered to the patient. It will be appreciated that the precise therapeutic dose will depend on the age and condition of the patient, nature of the condition to be treated and will be at the ultimate discretion of the attendant physician.

The term “excipient” or “pharmaceutically acceptable excipients” means a pharmacologically inactive component such as a diluent/filler, disintegrant, binder, surfactant, glidant, lubricant, plasticizer, opacifier, colorant, carrier, and the like, of a pharmaceutical product. The excipients that are useful in preparing a dosages form are generally safe, non-toxic and are acceptable for veterinary as well as human pharmaceutical use. Reference to an excipient includes both one excipient and more than one excipient.

The term “stable or stability or stabilized” means the dosages form are stable under accelerated conditions (40° C./75% RH) for at least six (06) months and are also stable under normal conditions (25° C./60% RH) for at least twelve (12) months. The term “composition” or “solid oral composition” or “dosage form” or “pharmaceutical composition” as used herein synonymously include tablet such as mono-layered tablets, bilayered tablets, trilayered tablet, multilayer tablet, caplets, minitablets, capsules, tablet in tablet, tablets in a capsule, granules in a capsule, pellets, pellets in a capsule, powder, granules, suspension or any other suitable dosage form meant for oral administration.

As used in this specification, the singular forms “a”, “an”, and “the” include plural references unless the context clearly dictates otherwise. Thus for example, a reference to “a method” or “a process” includes one or more methods, one or more processes and/or steps of the type described herein and/or which will become apparent to those persons skilled in the art upon reading this disclosure and so forth.

The term ‘extended release’ as used herein refers to specific release of drug over a specified time period, which may extend from about 2 hrs to about 24 hrs or more.

One aspect of the present invention provides an extended release oral pharmaceutical composition comprising a core, wherein the core comprises about 5-50% by weight of apremilast, at least one extended release polymer and one or more pharmaceutically acceptable excipients, wherein the extended release core is optionally coated with a film coating composition.

Another aspect of the present invention provides a pharmaceutical composition, wherein the at least one extended release polymer is selected from the group comprising of water insoluble polymers, water soluble polymers, pH dependent polymers, pH independent polymers and/or combinations thereof.

Another aspect of the present invention provides a pharmaceutical composition, wherein the pharmaceutical composition comprising about 5 mg to about 200 mg of apremilast or pharmaceutically acceptable salts, ester, solvates, polymorphs thereof. Preferably, the pharmaceutical composition contains about 5 mg to 100 mg of apremilast, most preferably 75 mg of apremilast.

Another aspect of the present invention provides a pharmaceutical composition, wherein the apremilast is present in any known polymorphic form i.e. amorphous or crystalline form.

Another aspect of the present invention provides a pharmaceutical composition, wherein the apremilast is present in any one of known polymorphic forms such as but not limited to, A, B, C, D, E, F, G, M, stereomerically pure, Form I, Form II, Form S, amorphous form, hydrates and solvate forms. Preferably, the apremilast is present in Form M and Form B.

Another aspect of the present invention provides a pharmaceutical composition, wherein the apremilast is having the particle size of d₉₀ less than about 200μ (microns).

Another aspect of the present invention provides a pharmaceutical composition, wherein the apremilast is having the particle size of d₉₀ less than about 100μ (microns).

Another aspect of the present invention provides an extended release oral pharmaceutical composition comprising (i) a core comprising apremilast or pharmaceutically acceptable salts, ester, solvates, polymorphs thereof, at least one extended release polymer and one or more pharmaceutically acceptable excipients; wherein the apremilast is being coated by or embedded in a matrix with one or more extended release polymer to achieve extended release; (ii) optionally a film coating.

Another aspect of the present invention provides an extended release oral pharmaceutical composition comprising apremilast, wherein the extended release may be achieved by one or more of coating or embedding in matrix using hydrophilic or hydrophobic polymers or by attachment to ion-exchange resins. Further, extended release may be achieved by osmotic oral release technology also.

Another aspect of the present invention provides a pharmaceutical composition, wherein the pharmaceutical composition is in the form of tablet, minitablet, granules, extrudes, pellets, powder, beads or spheroids, a microtablet, a capsule, granules in a capsule, pellets in a capsule, microtablets in a capsule and minitablets in a capsule.

Another aspect of the present invention provides a pharmaceutical composition, wherein the core is in the form of tablet, minitablet, granules, extrudes, pellets, powder, beads, spheroids, or a microtablet.

Another aspect of the present invention provides a pharmaceutical composition, wherein the pharmaceutical composition is suitable for oral, parenteral, transdermal, mucosal, nasal, buccal, or sublingual administration to a patient.

Another aspect of the present invention provides a pharmaceutical composition, wherein the pharmaceutical composition is in the form of delayed release, sustained release, extended release, controlled release, modified release and/or combinations thereof.

Another aspect of the present invention provides a pharmaceutical composition, wherein the pharmaceutical composition can be formulated by any suitable granulation methods are known in the art such as wet granulation, direct compression, extrusion spheronization, dry granulation/roller compaction or melt granulation.

Another aspect of the present invention, there is provided an extended release pharmaceutical composition comprising apremilast or pharmaceutically acceptable salts, ester, solvates, polymorphs thereof, one or more extended release polymers and one or more pharmaceutically acceptable excipients, wherein the pharmaceutical composition is used for the treatment of disease or disorder selected from the group consisting of psoriasis, psoriatic arthritis, rheumatoid arthritis, Behcet's Disease, rheumatoid spondylitis, an arthritic condition, atopic dermatitis, and ulcerative colitis, treating or managing obesity or overweight.

Suitable extended release polymer, without limitation includes hydrophilic or hydrophobic polymers comprise one or more of polyvinyl acetate, cellulose acetate, cellulose acetate butyrate, cellulose acetate propionate, ethyl cellulose, a fatty acid, a fatty acid ester, an alkyl alcohol, a wax, xanthan gum, gellan gum, shellac, rosin, zein (prolamine from corn), povidone, kollidon SR (polyvinyl acetate and povidone), a poly(meth)acrylate, poly(ethylene oxide), polyuronic acid salts, cellulose ethers, xanthan gum, tragacanth gum, gum karaya, guar gum, acacia, gellan gum locust bean gum, alkali metal salts of alginic acid or pectic acid, sodium alginate, potassium alginate, ammonium alginate, polyethylene oxide, carbomer homopolymer, hydroxypropyl cellulose, hydroxy ethyl cellulose, hydroxypropyl methyl cellulose (Methocel K100LVDC2, Methocel E5, Methocel K 100M, K100M DC, Methocel K4M, E4M, K4M DC), sodium carboxymethyl cellulose (Cekol 200P) carboxyvinyl polymers, polymerized gelatin, shellac, methacrylic acid copolymer type C NF, cellulose butyrate phthalate, cellulose hydrogen phthalate, cellulose propionate phthalate, polyvinyl acetate phthalate (PVAP), cellulose acetate phthalate (CAP), cellulose acetate trimellitate (CAT), hydroxypropyl methylcellulose phthalate, hydrogenated castor oil, stearic acid, hydrogenated vegetable oil, glyceryl behenate, glyceryl monostearate, hydroxypropyl methylcellulose acetate, dioxopropyl methylcellulose succinate, carboxymethyl ethyl cellulose (CMEC), hydroxypropyl methylcellulose acetate succinate (HPMCAS), and acrylic acid polymers and copolymers like methyl acrylate, ethyl acrylate, methyl methacrylate and/or ethyl methacrylate with copolymers of acrylic and methacrylic acid esters (Eudragit NE, Eudragit RL, Eudragit RS) and/or combinations thereof and the like. Polymer may be used from 0.1-50% by weight of the composition. Preferably, the polymer is present from 15-50% by weight of the composition.

Another embodiment of the present invention provides an extended release oral pharmaceutical composition of apremilast, wherein the pharmaceutical composition is free of cationic extended release polymer which includes chitosan, Carbopol® and Eudragit® and the like thereof.

Another embodiment of the present invention provides an extended release oral pharmaceutical composition of apremilast, wherein the pharmaceutical composition is free of anionic extended release polymer which includes sodium alginate, sodium carboxymethyl cellulose, chondroitin sulfate, carrageenan, pectin, gelatin, hyaluronic acid, glucosaminoglycans, mucoploysaaharides and the like thereof.

Another embodiment of the present invention provides an extended release oral pharmaceutical composition of apremilast, wherein the pharmaceutical composition is free of ionic extended release polymer.

Another embodiment of the present invention provides an extended release oral pharmaceutical composition of apremilast, and a non-ionic extended release polymer. Preferably, the pharmaceutical composition contains HPMC.

Another embodiment of the present invention provides an extended release oral pharmaceutical composition of apremilast which is free of floating agent/effervescent agent which includes bicarbonates or carbonates of sodium, potassium and calcium.

Another embodiment of the present invention provides an extended release oral pharmaceutical composition of apremilast which either contains a single extended release polymer or combination of its different grades.

Another aspect of the present invention provides pharmaceutical composition, wherein the one or more pharmaceutically acceptable excipients are selected from the group comprising of diluent/filler, binder, disintegrant, surfactant, glidant, lubricant, coloring agent/colorant, opacifier, plasticizer and/or combinations thereof. Some of the excipients may have two or more functions at the same time.

Suitable fillers/diluents include, without limitation, starch, corn starch, potato starch, pregelatinized starch, dry starch, disaccharides, lactose, cellulose, cellulose derivatives, such as silicified microcrystalline cellulose, microcrystalline cellulose, mannitol, sorbitol, xylitol, trehalose, colloidal silica, sucrose or other sugars or sugar derivatives, calcium hydrogen phosphate, dicalcium phosphate, low-substituted hydroxypopyl cellulose, hydroxyethyl cellulose, hydroxypropyl methyl cellulose, and/or combinations thereof. When present, a filler may be employed in an amount ranging from about 10% to about 80%, preferably from about 20% to about 80% by weight of the tablet.

Suitable binders include, without limitation, microcrystalline cellulose, polyvinylpyrrolidone (PVP), such as e.g., PVP K 30 or PVP90F, polyethylene glycols (PEG), e.g., PEG 4000, hydroxypropylmethyl cellulose, hydroxypropyl cellulose, both preferably of medium to high viscosity, e.g. viscosity grades 3 or 6 cps, pregelatinized starch and/or combinations thereof. When present, a binder may be employed in an amount ranging from about 0.1% to about 20%, preferably from about 0.5% to about 15%, such as 1% to 10%, by weight of the tablet.

Suitable lubricants include, without limitation, zinc stearate, magnesium stearate, sodium stearyl fumarate, aluminum or calcium silicate, stearic acid, PEG, talc and/or combinations thereof. When present, a lubricant may be employed in an amount ranging from about 0.01% to about 10%, preferably from about 0.1% to about 5%, by weight of the tablet.

Suitable disintegrants include, without limitation, carboxymethylcellulose calcium (CMC-Ca), carboxymethylcellulose sodium (CMC-Na), crosslinked PVP (e.g. crospovidone, polyplasdone XL or kollidon CL), croscarmellose sodium, alginic acid, sodium alginate and guar gum, most preferably crosslinked PVP (crospovidone), crosslinked CMC (Ac-Di-Sol), carboxymethyl starch-Na (pirimojel and explotab) and/or combinations thereof. A disintegrant is employed in an amount of 0.01 to 15%, such as of 0.05 to 12%, such as at least 0.1 to 10%, by weight of the tablet.

Suitable glidants include, without limitation, zinc stearate, colloidal silicon dioxide (e.g., Aerosil 200), magnesium trisilicate, powdered cellulose, starch, talc and/or combinations thereof. When present, a glidant may be employed in an amount ranging from about 0.01% to about 10%, preferably from about 0.1% to about 5%, by weight of the tablet.

The surfactants include but are not limited to anionic, cationic, non-ionic or amphoteric surfactants or those known to the person skilled in the art. Suitable surface active are poloxamer, polysorbate, cremophore, soluplus, lecithin and sodium lauryl sulfate and/or combinations thereof.

The pharmaceutical composition of the present invention may be further film coated. Film coating may be an immediate release or an extended release coating. The extended release coating comprises at least one or more extended release polymer and one or more pharmaceutically acceptable excipients.

Suitable immediate release film forming agents include, for example, polyvinylpyrrolidone, natural gums, starches, and cellulosic polymers. Examples of cellulosic polymers include, but are not limited to, hydroxypropyl methyl cellulose (“HPMC”), carboxymethyl cellulose (“CMC”) or salts thereof, hydroxypropyl cellulose (“HPC”), methylcellulose (“MC”), hydroxyethyl cellulose (“HEC”), and the like. Further, commercially available coating materials are available marketed under the brand name Opadry®. The polymers such as polyvinyl pyrrolidone, polyvinyl alcohol, polyvinyl acetate can be used as a film forming agent. In one aspect, the film forming agent can be used in an amount from 1% to 10% by weight of the composition.

Coloring agents include any FDA approved color for oral use.

Suitable plasticizers are selected from the group comprising of triethylcitrate, dibutyl sebacate, acetylated triacetin, tributylcitrate, glycerlotributyrate, monoglyceride, olive oil, sesame oil, acetyltributylcitrate, acetyltriethylcitrate, glycerin, sorbitol, diethyl oxalate, diethyl phthalate, diethyl malate, diethyl fumarate, dibutyl succinate and/or combinations thereof.

Suitable opacifiers are selected from the group comprising of titanium dioxide, manganese dioxide, iron oxide, silicon dioxide and/or combinations thereof.

Another aspect of the present invention provides an extended release oral pharmaceutical composition characterised by about 10-30% of apremilast is released within 2 hours; about 50-75% of apremilast is released within 8 hours and up to 95% of apremilast is released within 24 hours when dissolved in 2% Tween 80 in 50 mM sodium acetate buffer pH 4.5.

Another aspect of the present invention provides a method of treating or preventing a disease or disorder ameliorated by the inhibition of TNF-[α] production.

The present invention is further illustrated by the following examples which are provided merely to be exemplary of the invention and don't limit the scope of the invention. Certain modifications and equivalents will be apparent to those skilled in the art and are intended to be included within the scope of the present invention.

### Example 1—Extended Release Pharmaceutical Composition of Apremilast

| S. No. | Ingredient | mg | mg | mg | mg | mg |
|---|---|---|---|---|---|---|
| 1 | Apremilast | 75.0 | 75.0 | 75.0 | 75.0 | 75.0 |
| 2 | Microcrystalline | 150.0 | 150.0 | 150.0 | 150.0 | 150.0 |
| | Cellulose | | | | | |
| 3 | Kollidon SR | 150.0 | 150.0 | 150.0 | 150.0 | 150.0 |
| 4 | Sodium lauryl sulphate | 15.0 | — | — | — | — |
| 5 | Polysorbate | — | 15.0 | — | — | — |
| 6 | Poloxamer | — | — | 15.0 | — | — |
| 7 | Cremophore | — | — | — | 15.0 | — |
| 8 | Soluplus | — | — | — | — | 15.0 |
| 9 | Pregelatinised Starch | 100.0 | 100.0 | 100.0 | 100.0 | 100.0 |
| 10 | Colloidal Silicon Dioxide | 10.0 | 10.0 | 10.0 | 10.0 | 10.0 |
| 11 | Magnesium Stearate | 5.0 | 5.0 | 5.0 | 5.0 | 5.0 |

Film coating

| | | | | | | |
|---|---|---|---|---|---|---|
| 12 | Opadry | 12.5 | 12.5 | 12.5 | 12.5 | 12.5 |

Process:

(a) Dispense, sift and blend the apremilast, extended release polymer and other ingredients.

(b) Lubricate the blend obtained from step (a) with lubricants.

(c) Compress the lubricated blend using suitable punches and dies.

(d) Coated the compress mixture obtained in step (c) with coating solution.

### Example 2—Extended Release Pharmaceutical Composition of Apremilast

| S. | | | | | | |
|---|---|---|---|---|---|---|
| No. | Ingredient | mg | mg | mg | mg | mg |
| 1 | Apremilast | 75.0 | 75.0 | 75.0 | 75.0 | 75.0 |
| 2 | Microcrystalline | 150.0 | 150.0 | 150.0 | 150.0 | 150.0 |
| | Cellulose | | | | | |
| 3 | Sodium Alginate | 150.0 | 150.0 | 150.0 | 150.0 | 150.0 |
| 4 | Sodium lauryl sulphate | 15.0 | — | — | — | — |
| 5 | Polysorbate | — | 15.0 | — | — | — |
| 6 | Poloxamer | — | — | 15.0 | — | — |
| 7 | Cremophore | — | — | — | 15.0 | — |
| 8 | Soluplus | — | — | — | — | 15.0 |
| 9 | Pregelatinised Starch | 100.0 | 100.0 | 100.0 | 100.0 | 100.0 |
| 10 | Colloidal Silicon | 10.0 | 10.0 | 10.0 | 10.0 | 10.0 |
| | Dioxide | | | | | |
| 11 | Magnesium Stearate | 5.0 | 5.0 | 5.0 | 5.0 | 5.0 |

Film coating

| | | | | | | |
|---|---|---|---|---|---|---|
| 12 | Opadry | 12.5 | 12.5 | 12.5 | 12.5 | 12.5 |

Process: The pharmaceutical composition was prepared by the process as described in Example 1.

### Example 3—Extended Release Pharmaceutical Composition of Apremilast

| S. | | | | | | |
|---|---|---|---|---|---|---|
| No. | Ingredient | mg | mg | mg | mg | mg |
| 1 | Apremilast | 75.0 | 75.0 | 75.0 | 75.0 | 75.0 |
| 2 | Microcrystalline | 150.0 | 150.0 | 150.0 | 150.0 | 150.0 |
| | Cellulose | | | | | |
| 3 | Carbomer homopolymer | 150.0 | 150.0 | 150.0 | 150.0 | 150.0 |
| 4 | Sodium lauryl sulphate | 15.0 | — | — | — | — |
| 5 | Polysorbate | — | 15.0 | — | — | — |
| 6 | Poloxamer | — | — | 15.0 | — | — |
| 7 | Cremophore | — | — | — | 15.0 | — |
| 8 | Soluplus | — | — | — | — | 15.0 |
| 9 | Pregelatinised Starch | 100.0 | 100.0 | 100.0 | 100.0 | 100.0 |
| 10 | Colloidal Silicon | 10.0 | 10.0 | 10.0 | 10.0 | 10.0 |
| | Dioxide | | | | | |
| 11 | Magnesium Stearate | 5.0 | 5.0 | 5.0 | 5.0 | 5.0 |

Film coating

| | | | | | | |
|---|---|---|---|---|---|---|
| 12 | Opadry | 12.5 | 12.5 | 12.5 | 12.5 | 12.5 |

Process: The pharmaceutical composition was prepared by the process as described in Example 1.

### Example 4—Extended Release Pharmaceutical Composition of Apremilast

| S. No. | Ingredient | mg | mg | mg | mg | mg |
|---|---|---|---|---|---|---|
| 1 | Apremilast | 75.0 | 75.0 | 75.0 | 75.0 | 75.0 |
| 2 | Microcrystalline Cellulose | 150.0 | 150.0 | 150.0 | 150.0 | 150.0 |
| 3 | Xanthan or Gellan Gum | 150.0 | 150.0 | 150.0 | 150.0 | 150.0 |
| 4 | Sodium lauryl sulphate | 15.0 | — | — | — | — |
| 5 | Polysorbate | — | 15.0 | — | — | — |
| 6 | Poloxamer | — | — | 15.0 | — | — |
| 7 | Cremophore | — | — | — | 15.0 | — |
| 8 | Soluplus | — | — | — | — | 15.0 |
| 9 | Pregelatinised Starch | 100.0 | 100.0 | 100.0 | 100.0 | 100.0 |
| 10 | Colloidal Silicon Dioxide | 10.0 | 10.0 | 10.0 | 10.0 | 10.0 |
| 11 | Magnesium Stearate | 5.0 | 5.0 | 5.0 | 5.0 | 5.0 |

Film coating

| | | | | | | |
|---|---|---|---|---|---|---|
| 12 | Opadry | 12.5 | 12.5 | 12.5 | 12.5 | 12.5 |

Process: The pharmaceutical composition was prepared by the process as described in Example 1.

### Example 5—Extended Release Pharmaceutical Composition of Apremilast

| S. | | | | | | |
|---|---|---|---|---|---|---|
| No. | Ingredient | mg | mg | mg | mg | mg |
| 1 | Apremilast | 75.0 | 75.0 | 75.0 | 75.0 | 75.0 |
| 2 | Microcrystalline | 150.0 | 150.0 | 150.0 | 150.0 | 150.0 |
| | Cellulose | | | | | |
| 3 | Hydroxy propyl | 150.0 | 150.0 | 150.0 | 150.0 | 150.0 |
| | cellulose | | | | | |
| 4 | Sodium lauryl sulphate | 15.0 | — | — | — | — |
| 5 | Polysorbate | — | 15.0 | — | — | — |
| 6 | Poloxamer | — | — | 15.0 | — | — |
| 7 | Cremophore | — | — | — | 15.0 | — |
| 8 | Soluplus | — | — | — | — | 15.0 |
| 9 | Pregelatinised Starch | 100.0 | 100.0 | 100.0 | 100.0 | 100.0 |
| 10 | Colloidal Silicon | 10.0 | 10.0 | 10.0 | 10.0 | 10.0 |
| | Dioxide | | | | | |
| 11 | Magnesium Stearate | 5.0 | 5.0 | 5.0 | 5.0 | 5.0 |

Film coating

| | | | | | | |
|---|---|---|---|---|---|---|
| 12 | Opadry | 12.5 | 12.5 | 12.5 | 12.5 | 12.5 |

Process: The pharmaceutical composition was prepared by the process as described in Example 1.

### Example 6—Extended Release Pharmaceutical Composition of Apremilast

| S. | | | | | | |
|---|---|---|---|---|---|---|
| No. | Ingredient | mg | mg | mg | mg | mg |
| 1 | Apremilast | 75.0 | 75.0 | 75.0 | 75.0 | 75.0 |
| 2 | Microcrystalline | 150.0 | 150.0 | 150.0 | 150.0 | 150.0 |
| | Cellulose | | | | | |
| 3 | Polyethylene oxide | 150.0 | 150.0 | 150.0 | 150.0 | 150.0 |
| 4 | Sodium lauryl sulphate | 15.0 | — | — | — | — |
| 5 | Polysorbate | — | 15.0 | — | — | — |
| 6 | Poloxamer | — | — | 15.0 | — | — |
| 7 | Cremophore | — | — | — | 15.0 | — |
| 8 | Soluplus | — | — | — | — | 15.0 |
| 9 | Pregelatinised Starch | 100.0 | 100.0 | 100.0 | 100.0 | 100.0 |
| 10 | Colloidal Silicon | 10.0 | 10.0 | 10.0 | 10.0 | 10.0 |
| | Dioxide | | | | | |
| 11 | Magnesium Stearate | 5.0 | 5.0 | 5.0 | 5.0 | 5.0 |

Film coating

| | | | | | | |
|---|---|---|---|---|---|---|
| 12 | Opadry | 12.5 | 12.5 | 12.5 | 12.5 | 12.5 |

Process: The pharmaceutical composition was prepared by the process as described in Example 1.

### Example 7—Extended Release Pharmaceutical Composition of Apremilast

| S. | | | | | | | |
|---|---|---|---|---|---|---|---|
| No. | Ingredient | mg | mg | mg | mg | mg | mg |
| 1 | Apremilast | 75.0 | 75.0 | 75.0 | 75.0 | 75.0 | 75.0 |
| 2 | Hydrogenated castor | 150.0 | 150.0 | 150.0 | 150.0 | 150.0 | 150.0 |
| | oil/Stearic acid/ | | | | | | |
| | Hydrogenated | | | | | | |
| | vegetable oil/ | | | | | | |
| | Glyceryl behenate/ | | | | | | |
| | Glyceryl | | | | | | |
| | monostearate | | | | | | |

Extra granular

| | | | | | | | |
|---|---|---|---|---|---|---|---|
| 3 | Microcrystalline | 150.0 | 150.0 | 150.0 | 150.0 | 150.0 | 150.0 |
| | Cellulose | | | | | | |
| 4 | Sodium lauryl | 15.0 | — | — | — | — | — |
| | sulphate | | | | | | |
| 5 | Polysorbate | — | 15.0 | — | — | — | — |
| 6 | Poloxamer | — | — | 15.0 | — | — | — |
| 7 | Cremophore | — | — | — | 15.0 | — | — |
| 8 | Soluplus | — | — | — | — | 15.0 | — |
| 9 | Lecithin | — | — | — | — | — | 15.0 |
| 10 | Pregelatinised Starch | 100.0 | 100.0 | 100.0 | 100.0 | 100.0 | 100.0 |
| 11 | Colloidal Silicon | 10.0 | 10.0 | 10.0 | 10.0 | 10.0 | 10.0 |
| | Dioxide | | | | | | |
| 12 | Magnesium Stearate | 5.0 | 5.0 | 5.0 | 5.0 | 5.0 | 5.0 |

Film coating

| | | | | | | | |
|---|---|---|---|---|---|---|---|
| 13 | Opadry | 12.5 | 12.5 | 12.5 | 12.5 | 12.5 | 12.5 |

Process:

(a) Apremilast and one or more extended release polymer were mixed in blender and then melt extruded to form extrudates, which were cooled and milled to form granules.

(b) The granules obtained in step (a) were blended with one or more pharmaceutically acceptable excipients in blender to form uniform mixture.

(c) The mixture obtained in step (b) was compressed into the tablet which is further film coated using Opadry.

### Example 8—Extended Release Pharmaceutical Composition of Apremilast

| S. No. | Ingredient | mg | % w/w |
|---|---|---|---|
| 1 | Apremilast | 75.00 | 18.75 |
| 2 | Microcrystalline Cellulose | 142.00 | 35.50 |
| 3 | Hypromellose (High viscosity grade) | 18.75 | 4.69 |
| 4 | Hypromellose (Low viscosity grade) | 56.25 | 14.06 |
| 5 | Colloidal Silicon Dioxide | 2.00 | 0.50 |
| 6 | Magnesium Stearate | 1.00 | 0.25 |

Extragranular

| | | | |
|---|---|---|---|
| 7 | Mannitol | 100.00 | 25.00 |
| 8 | Colloidal Silicon Dioxide | 2.00 | 0.50 |
| 9 | Magnesium Stearate | 3.00 | 0.75 |
| | Total | 400.00 | 100.00 |

Process:

(a) Apremilast and one or more extended release polymer were mixed with one or more pharmaceutically acceptable excipients in blender to form uniform mixture which is compacted using roller compactor to obtain mass/ribbons which were milled and sized to obtain granules.

(b) The granules obtained in step (a) were blended with one or more pharmaceutically acceptable excipients in blender to form uniform mixture.

(c) The mixture obtained in step (b) was compressed into the tablet which may be optionally coated with film coating.

### Example 9—Extended Release Pharmaceutical Composition of Apremilast

| S. No. | Ingredient | mg/tab |
|---|---|---|
| 1 | Apremilast | 75.00 |
| 2 | Microcrystalline Cellulose | 152.00 |
| 3 | Colloidal Silicon Dioxide | 1.00 |
| 4 | Magnesium Stearate | 1.00 |

Extragranular

| | | |
|---|---|---|
| 5 | Microcrystalline Cellulose | 31.00 |
| 6 | Hypromellose (High viscosity grade) | 50.00 |
| 7 | Mannitol | 75.00 |
| 8 | Hypromellose (Low viscosity grade) | 110.00 |
| 8 | Colloidal Silicon Dioxide | 2.00 |
| 9 | Magnesium Stearate | 3.00 |
| | Total weight | 500.00 |

Process:

(a) Apremilast and one or more extended release polymer were mixed with one or more pharmaceutically acceptable excipients in blender to form uniform mixture which is compacted using roller compactor to obtain mass/ribbons which were milled and sized to obtain granules.

(b) The granules obtained in step (a) were blended with one or more extended release polymer and one or more pharmaceutically acceptable excipients in blender to form uniform mixture.

(c) The mixture obtained in step (b) was compressed into the tablet which may be optionally coated with film coating.

### Example 10—Extended Release Pharmaceutical Composition of Apremilast

| S. No. | Ingredient | mg/tab |
|---|---|---|
| 1 | Apremilast | 75.00 |
| 2 | Microcrystalline Cellulose | 178.00 |
| 3 | Colloidal Silicon Dioxide | 1.00 |
| 4 | Magnesium Stearate | 1.00 |

Extragranular

| | | |
|---|---|---|
| 5 | Sodium Carboxymethyl cellulose | 190.00 |
| 6 | Colloidal Silicon Dioxide | 2.00 |
| 7 | Magnesium Stearate | 3.00 |
| | Total weight | 450.00 |

(a) Apremilast and one or more pharmaceutically acceptable excipients were mixed in blender to form uniform mixture which is compacted using roller compactor to obtain mass/ribbons which were milled and sized to obtain granules.

(b) The granules obtained in step (a) were blended with one or more extended release polymer and one or more pharmaceutically acceptable excipients in blender to form uniform mixture.

(c) The mixture obtained in step (b) was compressed into the tablet which may be optionally coated with film coating.

Dissolution Study

The in vitro release study for the example 9 and 10 was carried out in 900 ml of 50 mM acetate buffer solution (pH 4.5) having 2% Tween 80, USP Type II (Paddle) apparatus at a speed of 75 rpm and 37° C. The dissolution data is given in Table 1.

TABLE 1
Percentage drug release
Dissolution conditions:
50 mM pH 4.5 AB + 2% Tween 80,
Volume 900 ml, 75 rpm,
USP II (Paddle)

| Time | % Drug release | |
|---|---|---|
| (Hours) | Example 9 | Example 10 |
| 1 | 9 | 4 |
| 2 | 20 | 14 |
| 4 | 40 | 33 |
| 6 | 56 | 52 |
| 8 | 69 | 64 |
| 12 | 79 | 75 |
| 16 | 84 | 80 |
| 24 | 88 | 85 |

Other embodiments of the invention will be apparent to those skilled in the art from consideration of the specification and practice of the invention disclosed herein. It is intended that the specification and examples be considered as exemplary only.

1. An extended release oral pharmaceutical composition comprising:
(i) a core comprising apremilast or its pharmaceutically acceptable salts, ester, solvates, polymorphs thereof, one or more extended release polymer and one or more pharmaceutically acceptable excipients; and
(ii) optionally a film coating.

2. The extended release oral pharmaceutical composition as claimed in claim 1, wherein said core comprises apremilast embedded in one or more extended release polymer.

3. The extended release oral pharmaceutical composition as claimed in claim 1, wherein said core comprises apremilast coated with one or more extended release polymer.

4. The extended release oral pharmaceutical composition as claimed in claim 1, wherein said core is free of cationic extended release polymer.

5. The extended release oral pharmaceutical composition as claimed in claim 1, wherein said pharmaceutical composition comprises about 5 mg to about 100 mg of apremilast.

6. The extended release oral pharmaceutical composition as claimed in claim 1, wherein one or more pharmaceutically acceptable excipients are selected from the group comprising of diluent/filler, binder, disintegrant, surfactant, glidant, lubricant, film forming polymer, coloring agent/colorant, opacifier, plasticizer and/or combinations thereof.

7. The extended release oral pharmaceutical composition as claimed in claim 1, wherein said pharmaceutical composition releases about 10-30% of apremilast within 2 hours; about 50-75% of apremilast is released within 8 hours and up to 95% of apremilast is released within 24 hours.

8. The pharmaceutical composition as claimed in claim 1, wherein said pharmaceutical composition is useful for treating or preventing a disease or disorder ameliorated by the inhibition of TNF-[α] production.

9. A process for preparation of extended release oral pharmaceutical composition comprising:
(i) a core comprising apremilast or its pharmaceutically acceptable salts, ester, solvates, polymorphs thereof, one or more extended release polymer and one or more pharmaceutically acceptable excipients; and
(ii) optionally a film coating, wherein said process comprising the following steps: (a) blending apremilast, one or more extended release polymers and one or more pharmaceutically acceptable excipients; (b) lubricating the blend obtained from step (a) with lubricants; (c) compressing the lubricated blend to form core; (d) optionally film coating the core with coating material.

10. The process for preparation of extended release oral pharmaceutical composition as claimed in claim 9, wherein said core is prepared by roller compaction or direct compression.
